(19) United States
(12) Patent Application Publication
(10) Pub. No.: US 2005/0287131 A1
(43) Pub. Date: Dec. 29, 2005

(54) Health supplement

(76) Inventor: Joel Floyd Schock, Fargo, ND (US)

Correspondence Address: Merchant & Gould P.C., P.O. Box 2903, Minneapolis, MN, 55402-0903, US

(21) Appl. No.: 10/877,332

(22) Filed: Jun. 25, 2004

(51) Int. Cl. A61K 35/78; A61K 38/43; A61K 35/80

(52) U.S. Cl. 424/94.1; 424/728; 424/195.17; 424/750; 424/755; 424/766; 424/764; 424/744; 424/767; 424/777

(57) ABSTRACT

The present invention relates to a health supplement that can include a number of ingredients that provide various health benefits. The supplement can be aloe vera based and include grape skin extract, grape seed extract, and/or citrus bioflavanoids. The supplement can also include a whole food component including ingredients such as spirulina, acelora berry, brown rice grain, and/or sunflower seed. The supplement can also include a cruciferous component, ginseng, metabolic coenzyme, and/or nopal cactus fiber. The supplement can also include a blend of amino acids, daily vitamins and minerals, and other beneficial ingredients. The supplement can also contain flavoring agents, preservatives, and a carrier, such as water.

### FIELD OF THE INVENTION

The present invention relates to a health supplement that can include a number of ingredients that provide various health benefits. The supplement can be aloe vera based. The supplement can include a bioflavanoid component that includes sources of bioflavanoids such as grape skin extract, grape seed extract, and/or citrus bioflavanoids. The supplement can also include a whole food component including ingredients such as spirulina, acelora berry, brown rice grain, and/or sunflower seed. The supplement can also include a cruciferous component, ginseng, a metabolic coenzyme, and/or nopal cactus fiber. The supplement can also include a blend of amino acids, daily vitamins and minerals, and other beneficial ingredients. The supplement can also contain flavoring agents, preservatives, and a carrier, such as water.

### BACKGROUND OF THE INVENTION

The diet of the average American lacks many essential vitamins, minerals, and nutrients. This can be attributed to numerous factors, primarily fast-paced, busy lifestyles that leave insufficient time to plan and prepare healthy meals. In addition, the vast number of fast foods and highly processed foods consumed by Americans prevent many people from consuming the basic nutrients a person needs.

As a person enters the elder stages of life he or she grows increasingly susceptible to diseases or disorders such as cardiovascular disease, tumor genesis, musculoskeletal deterioration, and many other diseases associated with aging. In order to prevent these diseases from developing later on in life, or at least reduce the impact made by such disorders, one should adopt a proper life style, including exercise and a well-rounded diet.

Moreover, the consuming public desires a health supplement comprised of naturally occurring ingredients. The demand for naturally occurring and/or organic products has grown immensely over the years. Thus, there is a desire for a health supplement based substantially, even entirely, on naturally occurring and/or naturally derived ingredients.

### SUMMARY OF THE INVENTION

The present invention relates to a health supplement. The supplement of the present invention can be used as a daily supplement to one's diet. The supplement can be aloe vera based, and can contain any of a number of beneficial or therapeutic ingredients. If desired, the supplement can be provided to include only naturally occurring and/or naturally derived components.

The supplement can include a bioflavanoid component, containing ingredients such as grape seed extract, grape skin extract, and/or citrus bioflavanoids. Some of the bioflavanoid sources provide other desirable substances as well. The supplement can include a cruciferous component, including ingredients such as broccoli and/or cauliflower extract. The supplement can include a whole food component which can include ingredients such as spirulina, acerola berry, brown rice grain, and/or sunflower seed. The supplement can also include ginseng, a metabolic coenzyme (such as coenzyme Q10), nopal cactus fiber, and/or an amino acid complex.

The supplement can be formulated to provide the recommended daily allowance (RDA) of vitamins and minerals, while also providing various other nutrients that have been shown to provide important health benefits. The supplement can be provided as a liquid, and can be formulated in order to provide a desired serving size, such as one fluid ounce.

### DETAILED DESCRIPTION OF THE PREFERRED EMBODIMENT

The present invention relates to a health supplement that can be used to supplement the diet with vitamins and minerals, while also providing various other nutrients that have been shown and are believed to provide important health benefits. The supplement can be provided in a formulation exhibiting organoleptic properties found desirable to consumers. Many of the amounts of substances are in fact limited by the desire to provide a supplement that consumers find tasteful.

Some ingredients can serve multiple functions, and some nutrients may be included for the same function as another nutrient. All the nutrients herein disclosed should be present in an amount sufficient to exert a therapeutic or taste-related effect without presenting any adverse health risks.

Many consumers desire products that are free from artificial ingredients. Accordingly, the supplement can also be provided using substantially only naturally occurring or naturally derived ingredients. The supplement can also be formulated to contain only natural occurring or naturally derived ingredients.

1. Aloe Vera

The supplement can contain aloe vera. In fact, the supplement can be based on aloe vera juice. It has long been believed that the aloe vera plant provides valuable health benefits. Historically, aloe vera has been used topically as a skin care product. It has also been used orally as a natural cathartic to reduce constipation. Aloe vera is believed to cleanse the digestive tract and allow for better digestion of nutrients. Aloe vera also exhibits a powerful anti-inflammatory effect by both reducing prostaglandins and decreasing neutrophil activity. Inflammation causes pain, swelling, and over time, inflammation can cause tissue damage. Aloe can be used to prevent such tissue damage by reducing inflammation throughout the body.

Researchers have found that aloe vera exhibits anti-oxidative effects and also lowers the cholesterol. (Lim et al., Efficacy of Dietary Aloe vera Supplementation on Hepatic Cholesterol and Oxidative Status in Aged Rats, Nutr. Sci. Vitaminol (Tokyo), August 2003, 49(4):292-96). It is believed that anti-oxidants stabilize free radicals and thereby help to prevent disease. As the body processes chemicals, the chemical reactions (typically involving oxygen) can cause molecules with unpaired electrons to develop. These molecules are called free radicals. Free radicals are quite unstable and quickly react with nearby molecules. For instance, it is believed that free radicals present in the body will react with proteins and enzymes. It is believed that this reaction damages proteins and enzymes, and can ultimately lead to cancer and various other diseases associated with the aging process, including heart disease and cataracts. Antioxidants react with free radical molecules to eliminate any unpaired electrons, and stabilize the free radicals. As a result, it is believed that antioxidants can prevent cancer and other maladies attributable to the aging process.

Aloe vera contains many substances that have been found to exhibit beneficial health effects. For instance, aloe vera contains aloe-emodin, a substance that is believed to have an anticancer effect. (Nordenberg et al., Combined Effect of Aloe-Emodin and Chemotherapuetic Agents on the Proliferation of an Adherent Variant Cell Line of Merkel Cell Carcinoma, Oncol. Rep., January 2004, 11(1):231-17); (Pecere et al., Involvement of p53 in Specific Anti-Neuroectodermal Tumor Activity of Aloe-Emodin, Int. J. Cancer, Oct. 10, 2003 106(6):836-47).

Aloe vera promotes tissue elasticity and healing by modulating cells critical to the healing process. Aloe vera also contains substances that aid in the healing of wounds. (Moon et al., A Novel Angiogenic Factor Derived From Aloe vera Gel: Betasitosterol, A Plant Sterol, Angiogenesis, 1993, 3(2):117-23).

By aggregating the above-offered benefits, aloe vera is believed to be able to slow the aging process or otherwise inhibit the development of age-related diseases.

Various forms of aloe vera are available, and the supplement can contain varying amounts of aloe vera depending on which form of aloe vera is used. Any commercially available, ingestible form of aloe vera can be used in the supplement. Aloe vera isolated from the inner filet of the aloe vera leaf is believed to provide the best source of health nutrients. Accordingly, the supplement can contain aloe vera isolated from this portion of the aloe vera leaf.

When included, the supplement should contain a sufficient amount of aloe vera to obtain the desired anti-oxidant activity. When the supplement is provided as an aloe-vera based composition, the supplement can comprise at least about 30 wt. % aloe vera. In order to provide an adequate amount of the remaining ingredients, the supplement can contain less than about 70 wt. % aloe vera. The supplement can also comprise from about 40 wt. % to about 60 wt. % aloe vera. As previously discussed, numerous forms of aloe vera are available. The above values correspond to aloe vera that is essentially in its natural state. For example, the aloe vera could be dried aloe vera, or aloe vera in powder form, that has been fully reconstituted in water.

2. Bioflavanoid Component

The supplement can also contain a bioflavanoid component. Bioflavanoids can be found naturally from a variety of sources. For instance, citrus fruits and grapes contain bioflavanoids. Exemplary bioflavanoid sources also include grape skin extract and grape seed extract. Sources such as these can be used to form the bioflavanoid component, and can be incorporated into the supplement.

Bioflavanoids have been found to act as antioxidants. Numerous kinds of bioflavanoids are present in nature. For instance, while grape seed and grape skin extract contain some of the same anti-oxidants, it is believed each of these sources can provide different anti-oxidants as well. For instance, grape seeds contain high quantities of proanthocyanides, a flavanoid exhibiting strong antioxidant activity. (Negro, Tommasi L. Phenolic Compounds and Antioxidant Activity From Red Grape Marc Extracts, Bioesour. Technol., March 2003, 87(1):41-44).

It is believed bioflavanoids present in grape skin and grape seed extracts also inhibit platelette function, thereby lowering the risk of blood clots forming within blood vessels. (Shanmuganayagam et al., Grape See and Grape Skin Extracts Elicit a Greater Antiplatelet Effect When Used in Combination Than When Used in Individually in Dogs and Humans, J. Nutr., December 2002, 132(12):3592-98).

Some sources of bioflavanoids include other, noflavanoid forms of anti-oxidants as well. For instance, grape seed extract and grape skin extract contain rather high levels of certain non-flavanoid anti-oxidants. Researchers believe that these nonflavanoid antioxidants, specifically the component resveratrol, serve to inhibit the proliferation and metastasis of cancerous cells. (Miura et al., Hypolipidemic Action of Dietary Resveratrol, a Phytoalexin in Grapes and Red Wine, in Hepatoma-Bearing Rats, Life Sci., Aug. 1, 2003 73(11): 1393-400). Resveratrol is even believed to exhibit neuroprotective activity. Zhuang et al., Potential Mechanism by Which Resveratrol, a Red Wine Constituent, Protects Neurons, Ann. N.Y. Acad. Sci., May 2003, 993:276-86, 287-88). Some researches believe that the antioxidants present in grape skin extract may slow the progression of degenerative diseases such as Alzheimer's. (Russo et al., Red Wine Micronutrients as Protective Agents in Alzheimer-like induced insult, Life Sci., Apr. 11, 2003 72(21):2369-79).

Grape skin and grape seed are also believed to exhibit anti-inflammatory and anti-viral action. (Bhat et al., Biological Effects of Resveratrol, Antioxid. Redox. Signal, December 2001, 3(6):1041-64). Grape seed extracts contain substances that serve to increase the nitric oxide content in the lining of blood vessels. (Yu et al., Study of Anti-atherosclerosic Effect of Grape Seed Extract and Its Mechanism, August 2002, 31(4):263-65). This prevents fatty compounds from thickening arterial walls and otherwise protects blood vessels. Resveratrol has also been found to exert cardioprotective effects, especially following ischemia. (Pataki et al., Grape Seed Proanthocyanidins Improved Cardiac Recovery During Reperfusion After Ischemia in Isolated Rat Hearts, Am. J. Clin. Nutr., May 2002, 75(5):894-99). Resveratrol is the compound present in red wine which researchers believe is the reason wine has been found to reduce the risk of a heart attack. Thus, while grape skin extract and grape seed extract have been characterized as sources of bioflavanoids, these substances can be included in the supplement to obtain the benefit of compounds other than bioflavanoids as well.

For these reasons, a bioflavanoid component can be included in the supplement. Some sources of bioflavanoids, such as grape skin extract and grape seed extract, can also contain additional non-bioflavanoid compounds. When included, the supplement should include at least enough of the bioflavanoid component to obtain the desired anti-oxidant effect. Thus, when included, the composition can include at least about 0.1 wt. % bioflavanoid component. Excessive amounts of bioflavanoid component provide little benefit and merely increase the cost of the supplement. Overly high levels of grape skin and/or grape seed extract, or other bioflavanoids can also cause the supplement to exhibit an undesirable taste. Accordingly, the supplement can comprise less than about 3 wt. % bioflavanoid component. The supplement can also contain from about 0.3 wt. % to about 2 wt. % bioflavanoid component, or even from about 0.5 wt. % to about 1.5 wt. % bioflavanoid component.

The bioflavanoid component can include one or more bioflavanoid sources such as grape skin extract, grape skin extract, and/or citrus bioflavanoids. Some bioflavanoid sources affect the taste of the supplement more than others. In addition some bioflavanoid sources contain higher levels of bioflavanoids than others, while some bioflavanoid sources offer desirable non-bioflavanoid compounds not offered by others. Accordingly, the amount of each bioflavanoid source used in the supplement can vary. For instance, the supplement can contain from about 0.1 wt % to about 2 wt. % grape skin extract, or even about 0.3 wt. % to about 1.5 wt. % grape skin extract. The supplement can also contain from about 0.1 wt. % to about 2 wt. % grape seed extract, or about 0.3 wt. % to about 1.5 wt. % grape seed extract. The supplement can also contain from about 0.1 wt. % to about 1 wt. % citrus bioflavanoids, or about 0.2 wt. % to about 0.6 wt. % citrus bioflavanoids. Of course, the supplement can be free of any or all of these bioflavanoids sources as well.

3. Cruciferous Component

The supplement can include a cruciferous component. Cruciferous, or Brassica, vegetables contain nutrients that have been found to exhibit anti-oxidative effects. The group of cruciferous vegetables includes vegetables such as broccoli, brussel sprouts, cabbage, cauliflower, rutabaga, watercress, bok choy, kale, and turnips.

Cruciferous vegetables provides a good, natural source of nutrients and vitamins. For instance, cruciferous vegetables are a good source of vitamin K, which helps promote blood clotting. Cruciferous vegetables are also a good source for fiber, and can aid in cleansing of the digestive tract.

Cruciferous vegetables, such as broccoli, also provide a source of substances called isothyocyanates that are believed to induce activity of phase 2 enzymes. Phase 2 enzymes are cancer fighting substances that inhibit the proliferation of cancerous cells. (Seow et al., Dietary Isothiocyanates, Glutathione S-transferase Polymorphism and Colorectal Cancer Risk in the Singapore Chinese Health Study, Carcinogenesis, December 2002, 23(12):2055-61); (Firestone et al., Indole-3-carbinol and 3-3′-diindoylmethane Antiproliferative Signaling Pathways Control Cell-Cycle Gene Transcription in Human Breast Cancer Cells By Regulating Promoter-Sp1Transcription Factor Interactions, J. Nutr., July 2003, 133(7 Supp.): 2448S-2455S); Finley, J W, The Antioxidant Responsive Element (ARE) May Explain the Protective Effects of Cruciferous Vegetables on Cancer, Nutr. Rev., July 2003, 61(7):250-54).

The addition of selenium to broccoli is believed to enhance the anti-oxidative effect of the extract. Selenium enriched broccoli is believed to inhibit tumorogenesis. (Finley, J W, Reduction of Cancer Risk by Consumption of Selenium-Enriched Plants: Enrichment of Broccoli With Selenium Increases the Anticarcinogenic Properties of Broccoli, J. Med. Food., 2003 Spring, 6(1):19-26); (Davis et al., Effect of Selenium-Enriched Broccoli Diet on Differential Gene Expression in Min Mouse Liver (1,2), J. Nutr. Biochem., April 2003, 14(4):227-31). Thus, the supplement can include a selenium enriched cruciferous component. The supplement can also include cruciferous vegetable that has not been enriched with selenium. For instance, the supplement can contain broccoli, both enriched with selenium or not enriched with selenium.

Cruciferous vegetables can be obtained in a variety of forms. For instance, they can be purchased in pill form, in their natural state, or ground. They can also be dried or undried. For purposes of characterizing the invention, the following measurements correspond to cruciferous vegetable that have been ground and dried to form a powder.

When included in the anti-oxidant blend, enough of the cruciferous component should be used to obtain anti-oxidant activity or otherwise provide a desired amount of whole food nutrients and vitamins. Accordingly, the supplement can include at least about 0.05 wt. % cruciferous component. Too much of the cruciferous component, however, can result in an undesirable taste. Moreover, once a threshold level of the cruciferous component has been included, additional cruciferous component does not offer much of an advantage, and results in unnecessary expenditure. Accordingly, the supplement can contain less than about 1 wt. % cruciferous component. The supplement can also contain from about 0.1 wt. % to about 0.5 wt. % cruciferous component. The cruciferous component can comprise a blend of cruciferous vegetables, or it can comprise a single cruciferous vegetable. For instance, the cruciferous component can comprise from about 0.1 wt. % to about 0.6 wt. % broccoli extract. Of course, the supplement can be provided free of the cruciferous component as well.

4. Whole Food Component

The supplement can also include a whole food component, including naturally occurring substances that provide good sources for vitamins, minerals, and nutrients. For instance, the supplement can include spirulina, acerola berry, brown rice grain, and/or sunflower seed. Spirulina is an algae and is a great natural source for a wide variety of vitamins. In order to obtain a supplement containing a larger variety of vitamins and minerals, the whole food component can include a blend of ingredients. For instance, the whole food component can include a blend of more than one of the following: spirulina, acerola berry, brown rice grain, and/or sunflower seed. The blend can contain equal amounts of these ingredients, or is can contain differing amounts of these ingredients. Of course, the whole food component can alternatively include only one of these compounds.

When the whole food component is included in the supplement, the supplement can comprise at least about 0.01 wt. % whole food component. Too much whole food component can result in an undesirable taste. Thus, the supplement can comprise up to about 1.2 wt. % whole food component. The supplement can also comprise from about 0.02 wt. % to about 0.5 wt. % whole food component, or even from about 0.03 wt. % to about 0.06 wt. % whole food component. The supplement can also be provided without a whole food component. These values correspond to the whole food substances in a dry, powdery form.

5. Ginseng

The anti-oxidant blend can also optionally include ginseng. For over two thousand years, Asians have recognized the therapeutic power of ginseng extract. While ginseng is believed to provide many different health benefits, one of the main benefits relevant to the supplement of the present invention is the ability of ginseng extract to increase the body's sensitivity to insulin. (Attele et al., Antidiabetic Effects of Panax Ginseng Berry Extract and the Identification of an Effective Component, Diabetes, June 2002, 51(6):1851-58)

One of the mechanisms believed to cause aging involves the decline in tissue function caused by the attachment of glucose molecules to proteins. This is especially a problem for diabetic individuals. A repeated exposure to high glucose levels causes accelerated damage to the vascular system, the connective tissue, and the immune system. For diabetics, this can cause heart disease, poor blood circulation (especially at the extremities of the body), and decreased joint flexibility. For non-diabetics, high glucose levels can damage proteins and contribute to connective tissue dysfunction, joint and muscle pain, joint and muscle stiffness, bursitis, and other maladies which exhibit an increased prevalence in middle-aged persons. Ginseng addresses these problems by rendering the body more sensitive to insulin while promoting weight loss. It is also believed ginseng can help to increase and/or moderate energy levels. Moreover, consumers find ginseng to be a desirable, naturally-occurring substance.

Numerous forms of ginseng can be included in the supplement. For instance, the Applicant has found that Panax ginseng can be included in the supplement. Enough ginseng should be present to provide an anti-oxidant effect. Alternatively, enough ginseng should be present to increase the user's sensitivity to insulin. Accordingly, when ginseng is used, the supplement can comprise at least about 0.05 wt. % ginseng. There is no real limit on how much ginseng can be included, however, high levels of ginseng provide only marginal benefit and simply increase the cost of the supplement. Thus, the supplement can contain less than about 1 wt. % ginseng. The supplement can also comprise about 0.1 wt. % to about 0.5 wt. % ginseng. While any of a number of forms can be used, the above values correspond to ginseng extract in dry, powder form.

6. Metabolic Coenzyme

The supplement can contact a metabolic coenzyme. The metabolic coenzyme can act as an anti-oxidant. It can also act as a digestive cofactor involved in electron transport, thereby supplementing the transfer of energy in cells. An exemplary metabolic coenzyme is coenzyme Q10. Coenzyme Q10 is an antioxidant that is believed to offer numerous health benefits. Studies have lead researchers to believe that coenzyme Q10 protects against congestive heart failure and improves blood circulation. (Overvad et al., Coenzyme Q10 in Health and Disease, Eur. J. Clin. Nutr., October 1999, 53(1):764-70). Researchers also believe that coenzyme Q10 protects against neurodegenerative diseases. (Beal et al., Coenzyme Q10 in the Central Nervous System and its Potential Usefulness in the Treatment of Neurodegenerative Diseases, Mol. Aspects Med., 1997 18 Supp.: S169-79). It has been found that as a person ages, his or her ability to synthesize this important enzyme from food decreases. (J. Hojerova, Coenzyme Q10—Its Importance, Properties and Use in Nutrition and Cosmetics, Ceska Slov. Farm., May 2000, 49(3):199-23). Researchers have determined that the ingestion of exogenous coenzyme Q10 increases the concentration of this enzyme in the heart and skeletal muscles, and in the mitochondria of brain cells. (Kamzalov et al., Coenzyme Q Intake Elevates the Mitochondrial and Tissue Levels of Coenzyme Q and Alpha-Tocopherol in Young Mice, J. Nutr., October 2003, 133(10):3175-80). In addition, no adverse side effects have been found to result from prolonged ingestion of this enzyme on a daily basis. (Overvad et al.).

The supplement can optionally include a metabolic coenzyme to obtain these and other desired benefits. If included, an amount of metabolic coenzyme sufficient to obtain these desired effects should be included. Thus, the supplement can comprise at least about 0.001 wt. % metabolic coenzyme. Excessive amounts of metabolic coenzyme result in waste. For this reason, the supplement can include up to about 0.1 wt. % metabolic coenzyme. The supplement can also contain from about 0.005 wt. % to about 0.05 wt. % metabolic coenzyme. Of course, the supplement can be provided without metabolic coenzyme as well.

As previously discussed, coenzyme Q10 is an exemplary metabolic coenzyme. An amount of coenzyme Q10 sufficient to obtain the desired effects should be included. Thus, the supplement can comprise at least about 0.001 wt. % coenzyme Q10. Excessive amounts of coenzyme Q10 results in waste. For this reason, the supplement can include up to about 0.1 wt. % coenzyme Q10. The supplement can also contain from about 0.005 wt. % to about 0.05 wt. % coenzyme Q10. Of course, the supplement can be provided without coenzyme Q10 as well.

7. Nopal Cactus Fiber

The supplement can also optionally include nopal cactus fiber. It is believed that nopal cactus fiber slows the adsorption of glucose from the gut, thereby lowering peak glucose effects. (Frat-Munari et al., Effect of a Dehydrated Extract of Nopal (Opuntia ficus indicia Mill.) on Blood Glucose, Arch. Invet. Med. (Mex.), July-September 1989, 20(3):211-16). Nopal cactus fiber is extracted from nopal cactus and is generally commercially available.

Enough nopal cactus fiber should be included to allow for glucose absorption by the stomach. Accordingly, when nopal cactus fiber is included in the supplement, the supplement can contain at least about 0.005 wt. % nopal cactus fiber. Once a threshold has been reached, however, additional nopal cactus fiber is wasteful and can have an adverse affect on the taste of the supplement. Accordingly, the supplement can contain up to about 0.05 wt. % nopal cactus fiber. The supplement can also contain from about 0.01 wt. % to about 0.03 wt. % nopal cactus fiber. These values correspond to nopal cactus fiber in a powder form.

8. Amino Acid Complex

The supplement can optionally include an amino acid complex. Amino acids are the building blocks of proteins, and are essential ingredients of the diet. The amino acid complex can include any of the amino acids, and can include them in virtually any form. The amino acid complex can comprise lysine, alanine, arginine, apartic acid, cystine, glutamic acid, glycine, histidine, isoleucine, leucine, methionine, phenylalanine, proline, serine, threonine, taurine, tyrosine, and valine. In other words, the supplement can include a blend of all 18 essential amino acids.

The supplement can include about 0.01 wt. % amino acid complex, if any is included. In order to avoid wasteful amounts of amino acid complex, the supplement can include less than about 1.2 wt. % amino acid complex. The supplement can also include from about 0.02 wt. % to about 0.5 wt. % amino acid complex, or even from about 0.03 wt. % to about 0.05 wt. % amino acid complex. These values correspond to an amino acid complex in powder form.

9. Daily Supplement Complex

The supplement can include a daily supplement complex. This complex can include supplements for a number of essential vitamins and minerals. The daily supplement blend can include those ingredients found in most daily vitamins. A list of exemplary ingredients of the daily supplement blend includes: Vitamin A, Vitamin C, Vitamin D, Vitamin E, Thiamin, Riboflvain, Niacin, Vitamin B6, Folic Acid, Vitamin B12, Biotin, Pantothenic Acid, Magnesium, Zinc, Selenium, Copper, Manganese, Chromium, and Molybdenum. It should be understood that different forms of these daily supplement ingredients can have differing bioavailabilities. Accordingly, some of the ingredients to the blend may be present in the same or higher amounts than those listed as the daily recommended allowance. Other ingredients may be provided at less that the daily recommended allowance.

The supplement can include any number of these standard vitamins and minerals. The supplement can comprise at least about 0.05 wt. % daily supplement complex. To avoid unnecessarily high levels of these vitamins and nutrients, the supplement can comprise less than about 1.5 wt. % daily supplement complex. The supplement can also comprise from about 0.3 wt. % to about 1.0 wt. % daily supplement complex. Of course, the supplement can be provided without the daily supplement complex.

10. Flavoring Agents

The supplement can also contain any one of a variety of known flavoring agents. The supplement can contain natural flavoring agents. Exemplary flavoring agents include plum extract, complex sugars such as fructose, and other flavoring agents known in the arts, such as polysaccharide. Artificial sweeteners can also be used. It may be desirable, however, for the supplement to be comprised substantially, even entirely, of naturally occurring and/or derived ingredients.

There is no real limit on the amount of flavoring agent that can be included other than taste and costs. Enough flavoring agent should be included, if any at all, to produce a supplement having a desirable taste. Conventional methods can be used to determine what flavoring agents, and levels thereof, should be included in the supplement. The composition can be provided free of flavoring agents as well.

11. Carrier

The supplement can optionally be diluted to a desired serving size using a carrier. The carrier can be any substance, such as a liquid, which can form a homogenous mixture of the supplement and can be dispensed in a manner to provide a predetermined serving size. The carrier should not interfere with any of the beneficial ingredients of the supplement so as to inhibit the desired activity. One exemplary carrier is water.

The amount of carrier is entirely within the discretion of the formulator, although higher levels of carrier may excessively dilute the supplement. In addition, certain levels of carrier may be used in order to obtain a desired serving size, a desired taste, or a desired texture or consistency. For example, the supplement can comprise at least about 20 wt. % carrier. To ensure an adequate amount of the remaining ingredients, the supplement should include less than about 50 wt. % carrier. The supplement can also comprise from about 30 wt. % to about 40 wt. % carrier, such as water.

The supplement can include a number of additional ingredients, such as preservatives, coloring agents, thickening agents, and other agents typically used in such supplements and known to those of skill in the art, that do not affect the activity of the above described active ingredients.

The supplement can be prepared for oral administration, and can be in the form of a liquid drink. The supplement can be formulated to be taken as a daily supplement. In addition, the supplement can be formulated to correspond to a desired serving size. For instance, the serving size can be at least about one-half fl. oz. The supplement can also be about 1 fl. oz. When the supplement is provided with a 1 fl. oz. serviing size, a full month supply could be sold in a single 32 fl. oz. bottle. These voumes correspond to the supplement in a drinkable fluid. The supplement could also be provided as a gel or encapsultated tablet in which case the serving size could be reduced to below one ounce, or even below one-half ounce.

It is contemplated that the consumer may shake the supplement prior to consumption in order to obtain a more homogenous mixture. It also is contemplated that the supplement could be diluted by the consumer. Of course, the supplement could also be diluted by the manufacturer. For instance, the supplement could be diluted and sold as a beverage, or it could be added to a beverage prior to sale.

When formulated for oral consumption, the supplement should be free from any substances that are unsuitable for human consumption. Substances that are typically present only in topically applied compositions that render the composition unsuitable for consumption can be eliminated from the supplement. For example, some substances that can be excluded from the composition include emollients, petroleum-based substances, and fragrances and perfumes that are unintended and unsuitable for human consumption.

The following table includes a few exemplary formulations of the health supplement of the current invention. These formulations are merely exemplary, and should not be construed as limiting the invention which is defined by the claims only.

| | Exemplary | Exemplary | Exemplary |
|---|---|---|---|
| | Supplement 1 | Supplement 2 | Supplement 3 |
| Component | (wt. %) | (wt. %) | (wt. %) |
| Aloe vera | 30-70 | 40-60 | 40-60 |
| Bioflavanoid | 0.1-3 | 0.3-2 | 0.5-1.5 |
| Component | | | |
| Grape seed extract | 0.1-2 | 0.3-1.5 | 0.3-1.5 |
| Grape skin extract | 0.1-2 | 0.3-1.5 | 0.3-1.5 |
| Citrus bioflavanoid | 0.1-1 | 0.2-0.6 | 0.2-0.6 |
| Cruciferous | 0.05-1 | 0.1-0.5 | 0.1-0.5 |
| Component | | | |
| Broccoli | 0.1-0.6 | 0.1-0.6 | 0.1-0.6 |
| Whole Food | 0.01-1.2 | 0.02-0.5 | 0.03-0.06 |
| Component | | | |
| Ginseng | 0.05-1 | 0.1-0.5 | 0.1-0.5 |
| Metabolic Coenzyme | 0.001-0.1 | 0.005-0.05 | 0.005-0.05 |
| Nopal cactus fiber | 0.005-0.05 | 0.01-0.03 | 0.01-0.03 |
| Amino acid complex | 0.01-1.2 | 0.02-0.5 | 0.03-0.05 |
| Daily Supp. Complex | 0.05-1.5 | 0.3-1.0 | 0.3-1.0 |
| Water | 20-50 | 30-40 | 30-40 |

The above specification, examples and data provide a complete description of the preparation and use of the supplement of the invention. Since many embodiments of the invention can be made without departing from the spirit and scope of the invention, the invention resides in the claims hereinafter appended.

1. A health supplement comprising:
at least about 30 wt. % aloe vera corresponding to aloe vera in its natural state;
at least about 0.1 wt. % bioflavanoid component;
at least about 0.05 wt. % cruciferous component.

2. The health supplement of claim 1 further comprising
at least about 0.1 wt. % whole food component, said whole food component comprising at least one of spirulina, acerola berry, brown rice grain, or sunflower seed.

3. The health supplement of claim 1, wherein the cruciferous component comprises at least one of broccoli or cauliflower.

4. (canceled)

5. The health supplement of claim 4 wherein the bioflavanoid component comprises at least one of grape seed extract, grape skin extract, or citrus bioflavanoid.

6. The health supplement of claim 1 further comprising
at least about 0.05 wt. % ginseng.

7. The health supplement of claim 1 further comprising
at least about 0.001 wt. % metabolic coenzyme.

8. The health supplement of claim 1 further comprising
at least about 0.005 wt. % nopal cactus fiber.

9. A health supplement comprising:
at least about 30 wt. % aloe vera corresponding to aloe vera in its natural state;
at least about 0.05 wt. % cruciferous component; and
at least about 0.1 wt. % whole food component, said whole food component comprising at least one of spirulina, acerola berry, brown rice grain, or sunflower seed.

10. The health supplement of claim 9, further comprising
at least about 0.1 wt. % bioflavanoid component.

11. The health supplement of claim 10 wherein the bioflavanoid component comprises at least one of grape seed extract, grape skin extract, or citrus bioflavanoid.

12. The health supplement of claim 9 wherein the supplement comprises at least about 30 wt. % aloe vera.

13. The health supplement of claim 9 wherein the cruciferous component comprises at least one of broccoli or cauliflower.

14. The health supplement of claim 9 further comprising
at least about 0.05 wt. % ginseng.

15. The health supplement of claim 9 further comprising
at least about 0.001 wt. % metabolic coenzyme.

16. The health supplement of claim 9 further comprising
at least about 0.005 wt. % nopal cactus fiber.

17. A health supplement comprising:
at least about 30 wt. % aloe vera corresponding to aloe vera in its natural state;
at least about 0.1 wt. % bioflavanoid component; and
at least about 0.1 wt. % whole food component, said whole food component comprising at least one of spirulina, acerola berry, brown rice grain, or sunflower seed.

18. (canceled)

19. The health supplement of claim 18 wherein the bioflavanoid component comprises at least one of grape seed extract, grape skin extract, or citrus bioflavanoids.

20. The health supplement of claim 17 further comprising
at least about 0.05 wt. % ginseng.

21. The health supplement of claim 17 further comprising
at least about 0.001 wt. % metabolic coenzyme.

22. The health supplement of claim 17 further comprising
at least about 0.005 wt. % nopal cactus fiber.
